SEQUENCE LISTING (1 sequence)

SEQ ID NO: 1 - protein, 9 aa, Artificial Sequence
  Synthetic Peptide
KVPRNQDWL

(19) United States
(12) Patent Application Publication
(10) Pub. No.: US 2018/0271870 A1
(43) Pub. Date: Sep. 27, 2018

(54) Compositions and Methods for Immune Therapy

(71) Applicant: Augusta University Research Institute, Inc., Augusta, GA (US)

(72) Inventors: Samir Khleif, Silver Spring, MD (US); Rasha Abu Eid, Martinez, GA (US)

(21) Appl. No.: 15/540,455

(22) PCT Filed: Dec. 30, 2015
(86) PCT No.: PCT/US2015/068061

§ 371 Date: Jun. 28, 2017

Related U.S. Application Data
(60) Provisional application No. 62/098,917, filed on Dec. 31, 2014.

(51) Int. Cl. A61K 31/519; A61K 31/4375; A61K 45/06

(52) U.S. Cl. CPC A61K 31/519; A61K 45/06; A61K 31/4375

(57) ABSTRACT

Inhibiting Akt1 and Akt2 but not Akt3 in a subject has been found to be an effective immune therapy that delays the exhaustion of CD8 T cells, prolongs CD8 T cell survival, preserves a remarkably high percentage of TCM cells, and significantly increases TCM proliferative potential upon reencountering antigen. In a preferred embodiment, the Akt1 and Akt2 inhibitors do not inhibit Akt3. Preferred small molecule inhibitors include, but are not limited to MK-2206, AZD5363, (1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate or combinations thereof.

### CROSS REFERENCE TO RELATED APPLICATIONS

This application claims priority to and benefit of U.S. Provisional Application No. 62/098,917 “Compositions And Methods for Immune Therapy” filed on Dec. 31, 2014, the disclosure of which is hereby incorporated herein by reference in their entirety.

### REFERENCE TO SEQUENCE LISTING

The Sequence Listing submitted Dec. 30, 2015 as a text file named “GRU_2015_004_PCT_ST25.txt”, created on Dec. 29, 2015, and having a size of 496 bytes is hereby incorporated by reference.

### FIELD OF THE INVENTION

The invention is generally directed to the field of immunology.

### BACKGROUND OF THE INVENTION

Upon antigen encounter and during the T cell response, CD8 T cells comprise effector and memory T cells (Kim, et al., Front Immunol, 4:20 (2013); Klebanoff, et al., Immunol Rev, 211:214-224 (2006)). While effector CD8 T cells become terminally differentiated and are eliminated by apoptosis following antigen clearance, approximately 10% of the remaining antigen-specific CD8 T cells become memory T cells (Klebanoff, et al., Immunol Rev, 211:214-224 (2006)). There are two types of CD8 memory T cells: central (TCM) and effector (TEM) memory T cells (Klebanoff, et al., Immunol Rev, 211:214-224 (2006); Sallusto, et al., Nature, 401:708-712 (1999)). Unlike TEM cells, TCM cells express a high level of CD62L and CCR7 and secrete high levels of IL-2, which correlates with their proliferative ability (Klebanoff, et al., Immunol Rev, 211:214-224 (2006); Sallusto, et al., Nature, 401:708-712 (1999); Klebanoff, et al., Proc Natl Acad Sci USA, 102:9571-9576 (2005)). It is therefore no surprise that TCM cells are superior in their ability to protect against viral and bacterial challenges when compared to TEM cells (Klebanoff, et al., Proc Natl Acad Sci USA, 102:9571-9576 (2005); Wherry, et al., Nat Immunol, 4:225-234 (2003)).

The quality of tumor antigen-specific CD8 T cells is crucial for an effective tumor immune response (Rosenberg, et al., J Immunol, 175:6169-6176 (2005)). Adoptive cell transfer (ACT) of tumor-reactive CD8 TCM cells has been shown to be a superior mediator of therapeutic antitumor immunity compared to TEM cells, due to their greater proliferative capacity upon antigen re-encounter (Klebanoff, et al., Proc Natl Acad Sci USA, 102:9571-9576 (2005); Wherry, et al., Nat Immunol, 4:225-234 (2003); Roberts, et al., J Exp Med, 202:123-133 (2005); Wu, et al., Cancer Lett (2013)). Accordingly, understanding the regulation of CD8 T cells into TEM or TCM cells is crucial, since defining a mechanism to enhance TCM cells and delay terminal differentiation of CD8 T cells can promote a better tumor immune response.

The duration and intensity of antigenic stimulation control the magnitude of the CD8 T cell response as well as their differentiation into effector and memory CD8 T cells. Agents that slow down the terminal differentiation of CD8 T cells without substantially impacting their proliferation are needed.

Therefore, it is an object of the invention to provide methods and compositions for inducing the formation of memory CD8 T cells.

It is another object of the invention to provide methods and compositions for modulating CD8 T cells to enhance CD8 T cell immune responses.

### SUMMARY OF THE INVENTION

It has been discovered that Akt1 and Akt2, but not Akt3, drive the terminal differentiation of CD8 T cells, and inhibition of Akt1 and Akt2 enhances the therapeutically superior central memory phenotype. Furthermore, the inhibition of Akt1 and Akt2, but not Akt 3, delays CD8 T cell exhaustion and preserves a reservoir of naïve and TCM CD8 T cells, thus enhancing their proliferative ability and survival and prolonging their cytokine production ability.

One embodiment provides methods of modulating CD8 T cells as part of different cancer immune therapy regimens. The CD8 T cells can be modulated by inhibiting Akt1, Akt2 or both. Representative inhibitors include, but are not limited to small molecule inhibitors such as MK-2206, AZD5363, (1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate or combinations thereof.

Other inhibitors of Akt1 and Akt2 can be used. These inhibitors include antisense oligonucleotides, ribozymes, siRNA, microRNA, antibodies specific for Akt1 and Akt2 or antigen binding fragments thereof.

Inhibition of Akt1 and Akt2 can be combined with other immune therapies including, but not limited to T cells genetically engineered to produce special receptors on their surface called chimeric antigen receptors (CARs) and other immune therapeutics such as anti-PD-L1 antibodies. The disclosed methods and compositions can be used to treat cancer, tumors and infections.

### BRIEF DESCRIPTION OF THE DRAWINGS

FIGS. 1A-1E show that Akt inhibition preserves the TCM phenotype and enhances the proliferative ability of CD8 T cells. Non-fractionated splenocytes from pMel-1 mice were stained with VCT and activated with gp100₂₅₋₃₃ peptide (1 μM) in the presence or absence of MK-2206 (0.67, 2 and 6 μM). The concentration of the inhibitors was maintained throughout the experiment. The cells were re-stimulated with gp100₂₅₋₃₃ peptide on days 7, 14 and 21 and their phenotype and proliferation of assessed. The gated cells were viable (7AAD−) CD8+Vβ13+. FIG. 1A shows CD8 T cells from a naïve spleen (far left) are mainly (72%) naïve cells (CD62LhiCD44lo). Sixty seven percent (67%) of non-MK-2206-treated CD8 T cells (3rd graph from left) are T_{EM} cells (CD62LloCD44hi). This changes when cells are treated with MK-2206 (far right), as 65% of the cells possess the TCM phenotype (CD62LhiCD44hi). FIG. 1B shows that after 3 days of stimulation, the proliferation of CD8 T cells was inhibited in a dose-dependent manner by MK-2206 (VCT dilution) (far left). CD8 T cells treated with MK-2206 expand at a significantly high rate with further stimulations (middle graph) (data normalized to the non-treated control (GP100). MK-2206-treated CD8 T cells secrete significantly higher levels of IL-2 following stimulations 2 and 3, which is consistent with their higher proliferative potential (far right). *p<0.05, ** p<0.01, ****p<0.0001. FIG. 1C shows that Akt inhibition by MK-2206 maintains a high level of CD62L expression in CD8 T cells on day 3, and on day 7 after each stimulation with gp100. FIG. 1D shows Akt inhibition by MK-2206 maintains high levels of CD127 in CD8 T cells on day 3, and on day 7 after each stimulation with gp100. FIG. 1E shows Akt inhibition by MK-2206 inhibits the up-regulation of the exhaustion marker KLRG-1 in CD8 T cells after the second and third stimulations with gp100.

FIGS. 2A and 2B show that Akt inhibition by MK-2206 maintains a high level of IFNγ and TNF secretion in CD8 T cells. CD8 T cells from pMel-1 mice were stimulated with gp10025-33 peptide (1 μM) in the presence or absence of MK-2206 (0.67 μM). On days 7 and 14, CD8 T cells were re-stimulated with gp10025-33 peptide and the IFNγ and TNF levels in the supernatant assessed after 24 hours using CBA. FIG. 2A shows the ability of CD8 T cells to produce IFNγ with subsequent stimulations is significantly diminished. CD8 T cells treated with MK-2206 maintain their ability to secrete IFNγ with further stimulations. *p<0.05,****p<0.0001. FIG. 2B shows that CD8 T cells treated with MK-2206 produce significantly higher levels of TNF and maintain this ability with further stimulations. *p<0.05.

FIGS. 3A-3E show the inhibition of Akt1 and Akt2 preserves TCM cells and enhances the proliferative ability of CD8 T cells. Non-fractionated splenocytes from pMel-1 mice were stained with VCT and activated with gp100₂₅₋₃₃ peptide (1 μM) in the presence or absence of Akt 1/2 inhibitor (2.2, 6.7 and 20.1 μM). The cells were re-stimulated with gp100₂₅₋₃₃ on days 7, 14 and 21. The gated cells were viable (7AAD−) CD8+Vβ13+. FIG. 3A shows Akt1 and Akt2 inhibition preserves the T_{CM} phenotype. In this representative example, seventy six percent (76%) of non-treated CD8 T cells are T_{EM} cells (CD62L^{lo}CD44^{hi}), while CD8 T cells treated with Akt 1/2 inhibitor consist of 76% T_{CM} cells (CD62L^{hi}CD44^{hi}). FIG. 3B shows the proliferation of CD8 T cells is inhibited by Akt 1/2 inhibitor in a dose-dependent manner (day3). The expansion of CD8 T cells treated with the inhibitor is significantly enhanced with further stimulations. Data is normalized to the non-treated control (GP100). * p<0.05. FIG. 3C shows Akt1 and Akt2 inhibition maintains a high level of CD62L on day 3, and on day 7 after each stimulation. FIG. 3D shows Akt1 and Akt2 inhibition maintains a high level of CD127 on day 3, and on day 7 after each stimulation. FIG. 3E shows Akt1 and Akt2 inhibition mitigates the up-regulation of KLRG-1 in CD8 T cells after the second and third stimulations.

FIGS. 4A-4C show the absence of Akt 1 and Akt 2 isoforms, but not Akt 3, preserves the TCM phenotype. Enriched CD8 T cells from Akt1, 2 and 3 KO and WT mice were stimulated with anti-CD3 (1 ng/ml) and co-stimulated with anti-CD28 (2.5 μg/ml) antibodies. The phenotype of the cells was assessed on day 7. The gated cells were viable (7AAD−) CD8+. FIG. 4A shows WT CD8 T cells consisted of 83% T_{EM} cells (CD62L^{lo}CD44^{hi}). Akt1 KO CD8 T cells consisted of 55% TCM cells (CD62L^{hi}CD44^{hi}), with 43% of the cells being TEM cells. Akt2 KO CD8 T cells consist of 36% T_{CM} and 63% T_{EM} cells. Akt3 KO CD8 T cells consisted mainly of 86% T_{EM} cells, while only 12% were T_{CM} cells. FIG. 4B shows Akt1KO cells express a higher level of CD62L than Akt2 KO cells, which in turn express higher levels of these markers than WT and Akt3 KO cells, which express similar levels. FIG. 4C shows Akt1 KO cells express a higher level of CD127 than Akt2 KO cells, which in turn express higher levels of these markers than WT and Akt3 KO cells, which express similar levels.

FIGS. 5A-5D show Akt inhibition preserves the T_{CM} phenotype in WT and Akt KO mice. Enriched CD8 T cells from Akt 1, 2 and 3 KO and WT mice were stimulated anti-CD3 (1 ng/ml) and co-stimulated with anti-CD28 (2.5 μg/ml) antibodies in the presence or absence of MK-2206 (0.67 μM), or Akt 1/2 inhibitor (2.2 μM). The phenotype of the cells was assessed on day 7. The gated cells were viable (7AAD−) CD8+. FIG. 5A shows untreated CD8 T cells from WT mice consist mainly of T_{EM} cells, while those treated with MK-2206 or Akt1/2 inhibitors consist mainly of T_{CM} cells. FIG. 5B shows CD8 T cells from Akt1 KO mice possess significantly more T_{CM} cells than WT without any inhibitors. Once treated with MK-2206 or Akt1/2 inhibitors, more TCM cells (with a higher CD62L expression) are maintained comparable to treated WT cells. FIG. 5C shows CD8 T cells from Akt2 KO mice possess significantly more T_{CM} cells than WT without any treatments, although less than that observed from Akt1KO mice. Treatment with MK-2206 or Akt1/2 inhibitors maintains a significantly higher percentage of T_{CM} cells comparable to WT and Akt1 KO treated cells. FIG. 5D show CD8 T cells from Akt3 KO mice consist mainly of T_{EM} cells. Treatment with MK-2206 or Akt1/2 inhibitors maintains a significantly higher percentage of T_{CM} cells comparable to WT and Akt1 and 2 KO treated cells.

FIGS. 6A-6E show that Akt inhibition preserves the T_{CM} phenotype and increases the proliferation of CD8 T cells. Non-fractionated splenocytes from pMel-1 mice were stained with VCT and activated with gp100₂₅₋₃₃ peptide (1 μM) in the presence or absence of AZD5363 (0.27, 0.81 and 2.4 μM). The cells were re-stimulated with gp100₂₅₋₃₃ on days 7, 14 and 21. The gated cells were viable (7AAD−) CD8+Vβ13+. FIG. 6A shows Akt inhibition by AZD5363 preserves the T_{CM} phenotype. In this representative example, 59% of non-treated CD8 T cells (GP100) are T_{EM} cells. Treatment with AZD5363 enhances the percentage of T_{CM} cells, where 91% of the cells possess the T_{CM} phenotype. FIG. 6B shows that Akt inhibition by AZD5363 inhibits the proliferation of CD8 T cells in a dose-dependent manner (day3, VCT dilution). Treatment with AZD5363 enhanced the proliferation of CD8 T cells with further stimulations. Data is normalized to the non-treated control (GP100). FIG. 6C shows Akt inhibition by AZD5363 maintains a high level of CD62L in CD8 T cells. This is observed on day 7 after each stimulation with gp100. FIG. 6D shows Akt inhibition by AZD5363 maintains a high level of CD127 in CD8 T cells. This is observed on day 7 after each stimulation with gp100. FIG. 6E shows Akt inhibition by AZD5363 inhibits the up-regulation of KLRG-1 in CD8 T cells after the second and third stimulations.

FIG. 7 shows Akt inhibition in CD8 T cells preserves the T_{CM} phenotype, which is persistent even after the second and third stimulation. Non-fractionated splenocytes from pMel-1 mice were activated with gp100₂₅₋₃₃ peptide (1 μM) in the absence or presence of MK-2206 (0.67 μM) or Akt 1/2 inhibitor (2.2 μM). Cells were harvested on day 7 after each stimulation. The gated cells were viable (7AAD−) CD8+Vβ13+. Without any treatment (far left panel), the reservoir of T_{CM} cells was gradually depleted with each stimulation, while a high percentage was preserved when the cells were treated with the inhibitors (middle and right panels).

### DETAILED DESCRIPTION OF THE INVENTION

### I. Methods and Compositions for Immune Therapy

A. Inhibition of Akt1 and Akt2

Inhibiting Akt1 and Akt2 but not Akt3 in a subject has been found to be an effective immune therapy that delays the exhaustion of CD8 T cells, prolongs CD8 T cell survival, preserves a remarkably high percentage of T_{CM} cells, and significantly increases T_{CM} proliferative potential upon reencountering antigen. In a preferred embodiment, the Akt1 and Akt2 inhibitors do not inhibit Akt3. Preferred small molecule inhibitors include, but are not limited to MK-2206, AZD5363, (1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate or combinations thereof.

In another embodiment, the inhibitors of Akt1 and Akt2 can be antisense oligonucleotides, ribozymes, siRNA, microRNA, antibodies specific for Akt1 and Akt2 or antigen binding fragments thereof.

One embodiment provides a method for maintaining a reservoir of central T memory cells in a subject by administering to the subject an effective amount of one or more inhibitors of Akt1 and Akt2 to enhance the proliferative potential, function, and survival of CD8 T cells. A single inhibitor of both Akt1 and Akt2 can be used or a combination of Akt1 inhibitor and an Akt2 inhibitor can be used together.

Another embodiment provides a method for modulating CD8 T cells to enhance proliferative potential, function and survival by contacting the CD8 T cells with an effective amount of one or more inhibitors of Akt1 and Akt2 to induce the formation of central T memory cells. Akt1 and Akt2 inhibition enhances the central memory phenotype of CD8 T cells by diminishing their terminal differentiation and increasing their proliferative ability and survival.

Still another embodiment provides a method for delaying T cell exhaustion by administering an effective amount of one or more Akt1 and Akt2 inhibitors to a subject in need thereof. Preferably the subject is a human.

Yet another embodiment provides a method for treating cancer or a tumor in a subject by administering to the subject an effective amount of one or more inhibitors of Akt1 and Akt2 to reduce tumor burden in the subject. Representative cancers include but are not limited to head and neck cancer, lung cancer, small cell carcinoma, colon cancer, stomach cancer, throat cancer, melanoma, sarcoma, and cancers of internal organs.

Another embodiment provides a method for maintaining a high expression of CD62L and CD127 on CD 8 T cells in a subject by administering and effective amount of one or more inhibitors of Akt1 and Akt2 to the subject.

Additionally, Akt1 and Akt 2 inhibitor can rescue the ability of CD8 T cells to secrete high levels of TNF and IFNγ secretion, even following multiple stimulations, thus suggesting a prolonged and potent anti-tumor cytotoxic ability.

The disclosed compositions and methods can be used to treat infections including viral and bacterial infections.

One embodiment provides a method for enhancing a vaccine by administering one or more inhibitors of Akt1 and Akt2 but not Akt3 to the subject in combination with the vaccine in an amount effective to enhance proliferative potential, function and survival of CD8 T cells in the subject.

It will be appreciated that the CD8 T cells can be contacted with Akt1 and Akt2 inhibitors either in vivo, in vitro or both.

B. Combination Therapies

The disclosed compositions and methods can be used in conjunction or alternation with other immune therapies. For example the disclosed compositions and methods can be used in conjunction with the administration of T cells genetically engineered to produce chimeric antigen receptors that target a specific antigen, for example a tumor antigen. Other additional therapies include anti-PD-L1 antibodies or other monoclonal antibody therapies.

The compositions can be combined with one or more additional therapeutic agents. Representative therapeutic agents include, but are not limited to chemotherapeutic agents and pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

The methods and compositions can be used in conjunction or alternation with an immune response stimulating agent. The immune system is composed of cellular (T-cell driven) and humoral (B-cell driven) elements. It is generally accepted that for cancer, triggering of a powerful cell-mediated immune response is more effective than activation of humoral immunity. Cell-based immunity depends upon the interaction and co-operation of a number of different immune cell types, including antigen-presenting cells (APC; of which dendritic cells are an important component), cytotoxic T cells, natural killer cells and T-helper cells. Therefore, the active agent can be an agent that increases a cell (T-cell driven) immune response, a humoral (B-cell driven) immune response, or a combination thereof. For example, in some embodiments, the agent enhances a T cell response, increases T cell activity, increases T cell proliferation, reduces a T cell inhibitory signal, enhances production of cytokines, stimulates T cell differentiation or effector function, promotes survival of T cells or any combination thereof.

Exemplary immunomodulatory agents include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone). In particular embodiments, the combination therapy includes cytokine-based immunotherapeutic agent, for example, interferon (e.g., IFN type I, II, or III), or interleukin (e.g, interleukin-2)

In some embodiments the agent is an inflammatory molecule such as a cytokine, metelloprotease or other molecule including, but not limited to, IL-1 (3, TNF-α, TGF-beta, IFN-γ, IL-17, IL-6, IL-23, IL-22, IL-21, and MMPs.

In some embodiments, the combination therapy include an antibody-based immunotherapeutic agent, for example, Alemtuzumab, Bevacizumab, Brentuximab vedotin, Cetuximab, Gemtuzumab ozogamicin, Ibritumomab tiuxetan, Ipilimumab, Ofatumumab, Panitumumab, Rituximab, Tositumomab, Trastuzumab, an anti-CD47 antibody, or an anti-GD2 antibody.

In some embodiments, the compositions are administered with PD-1 antagonists that bind to and block PD-1 ligands and thereby prevent them from interacting with PD-1. PD-1 antagonists that bind to and block endogenous PD-1 on immune cells, preferably T cells, include PD-L1 and PD-L2 polypeptides, PD-1-binding fragments thereof, PD-1 antibodies, fusion proteins, and variants thereof. These PD-1 antagonist bind to PD-1 under physiological conditions and block T cell inhibition.

PD-1 antagonists that bind to native PD-1 ligands include PD-1 and B7.1 polypeptides, fragments thereof, antibodies, and fusion proteins. These PD-1 antagonists bind to B7-H1 and B7-DC and prevent them from triggering inhibitory signal transduction through PD-1 on immune cells. An exemplary PD-L2-Ig fusion proteins are disclosed in WO 2010/027828.

In some embodiments, the compositions may be co-administered with compositions containing B7 family costimulatory molecules that enhance an immune response. The other B7 costimulatory polypeptide may be of any species of origin. In one embodiment, the costimulatory polypeptide is from a mammalian species. In a preferred embodiment, the costimulatory polypeptide is of murine or human origin. In a particular embodiment, the additional agent is B7.1. Other useful human B7 polypeptides can have at least about 80, 85, 90, 95 or 100% sequence identity to the B7-2 polypeptide encoded by the nucleic acid having GenBank Accession Number U04343; or the B7-H5 polypeptide encoded by the nucleic acid having GenBank Accession Number NP_071436.

In a preferred embodiment, the additional B7 family molecules are provided as soluble fusion proteins. Soluble fusion proteins of B7 molecules that form dimers or multimers and have the ability to crosslink their cognate receptors and thereby function as receptor agonists.

In preferred embodiments of any of the compositions are administered in combination with at least one additional agent selected from the group consisting of an anti-PD-1 antibody, an anti-B7-H1 antibody, an anti-CTLA4 antibody, a mitosis inhibitor, such as paclitaxel, an aromatase inhibitor, such as letrozole, an A2AR antagonist, an angiogenesis inhibitor, anthracyclines, oxaliplatin, doxorubicin, TLR4 antagonists, and IL-18 antagonists.

### II. Compositions for Decreasing the Bioactivity of Akt1 and Akt2

Compositions including one or more compounds for decreasing the bioactivity of Akt1 and Akt2 disclosed. In some embodiments, the compound is an inhibitory Akt1 or Akt2 polypeptide, an inhibitory fusion protein including an Akt1 or Akt2 polypeptide; a small molecule or peptidomimedic antagonist of Akt1 or Akt2, or an inhibitory nucleic acid that targets genomic or expressed Akt1 or Akt2 nucleic acids (e.g., Akt1 or Akt2 mRNA), or a vector that encode an inhibitory nucleic acid. The preferred embodiments, the inhibitory protein, peptidomimedic, or small molecule antagonist binds to blocks the catalytic domain of Akt1 or Akt2, or otherwise prevents Akt1 or Akt2 from binding to or to its substrate(s) or

In some embodiments the compound inhibits Akt1 and Akt2 without inhibiting Akt3. In preferred embodiments, the compound has a higher specificity, a higher affinity, or a combination thereof for Akt1 or Akt2 than for Akt3.

A. Inhibitory Akt1 and Akt2 Polypeptides

In some embodiments, the compound that decreases the bioavailability of Akt1 and/or Akt2 is an inhibitory polypeptide. Inhibitory polypeptides are typically non-functional fragments or variants of Akt1 or Akt2 or both. For example, an inhibitory Akt1 or Akt2 polypeptide can be a fragment or variant of Akt1 or Akt2 that can bind to an Akt1 or Akt2 substrate but has reduced kinase activity compared to endogenous Akt1 or Akt2, or preferably, does not phosphorylate the substrate. Therefore, in some embodiments, the inhibitory peptide competes with endogenous Akt1 or Akt2 for binding to Akt1 or Akt2 substrates, thereby reducing the bioavailability of the endogenous Akt1 or Akt2. Preferred inhibitory peptides bind to an Akt1 or Akt2 substrate and prevent binding of endogenous Akt1 or Akt2 from binding to and/or phosphorylating the substrate. In some embodiments, the inhibitor polypeptide binds to the substrate with higher affinity or specificity than Akt1 or Akt2.

For example in a preferred embodiment, the inhibitory peptide has one or more substitutions, deletions, or insertions in the kinase domain, C-terminal regulatory region, or a combination thereof that reduce the ability of Akt1 or Akt2 or both to be fully activated. In some embodiments the inhibitory polypeptide is a known variant or fragment of Akt1 or Akt2 that lacks kinase activity.

B. Small Molecules, Substrate Mimics, and

Peptidomimetics

1. Small Molecules

In some embodiments, the compound that reduces bioactivity of Akt1, Akt2 or both is a compound that binds to Akt1 or Akt2 and reduces or prevents kinase activity or the binding specificity or affinity of Akt1 or Akt2 or both to a substrate of Akt1 or Akt2 or both. In some embodiments, the compound is a small molecule. The term “small molecule” generally refers to small organic compounds having a molecular weight of more than about 100 and less than about 2,500 Daltons, preferably between 100 and 2000, more preferably between about 100 and about 1250, more preferably between about 100 and about 1000, more preferably between about 100 and about 750, more preferably between about 200 and about 500 Daltons. The small molecules can include cyclical carbon or heterocyclic structures and/or aromatic or polyaromatic structures substituted with one or more functional groups.

Small molecule inhibitors of Akt1 and Akt2 are known in the art and include, for example, MK-2206, AZD5363, Akt 1/2 inhibitor (1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate. Generally, the compounds can be administered to humans in an amount from about 0.0001 mg/kg of body weight to about 1,000 mg/kg of body weight per day. Generally, for intravenous injection or infusion, dosage may be lower than for other methods of delivery.

MK-2206 has the structure:

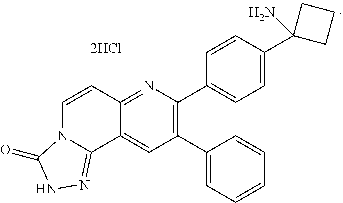 [SMILES: NC1(c2ccc(-c3nc4ccn5c(=O)[nH]nc5c4cc3-c3ccccc3)cc2)CCC1]

MK-2206 2HCl is a highly selective inhibitor of Akt1/2/3 with IC50 of 8 nM/12 nM/65 nM, respectively; no inhibitory activities against 250 other protein kinases observed.

MK-2206 2HCl has been administered at a dosage of 120 mg/kg and 240 mg/kg in a mouse model. In a first-in-man clinical trial of the oral pan-Akt inhibitor MK-2206 in patients with advanced solid tumors.

Thirty-three patients received MK-2206 at 30, 60, 75, or 90 mg on alternate days. Dose-limiting toxicities included skin rash and stomatitis, establishing the MTD at 60 mg. Therefore, in some embodiments, the MK-2206 2HCl compositions disclosed herein include 0.1 mg to 1,000 mg, preferably 1 mg to 500 mg, more preferably, 5 mg to 60 mg per day.

AZD5363 potently inhibits all isoforms of Akt(Akt1/Akt2/Akt3) with IC50 of 3 nM/8 nM/8 nM. AZD5363 has the structure:

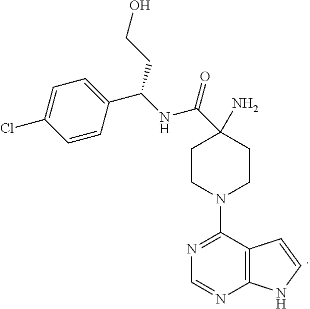 [SMILES: NC1(C(=O)N[C@@H](CCO)c2ccc(Cl)cc2)CCN(c2ncnc3[nH]ccc23)CC1]

AZD5363 has been administered orally at dosages of 100 mg/kg, 130 mg/kg, 200 mg/kg, and 300 mg/kg in a mouse model. In a clinical trial testing the effect of AZD5363 on patients with advanced solid tumors, an intermittent dosing schedule of 480 mg twice a day was generally well tolerated AACR Press Releases “Akt Inhibitor AZD5363 Well Tolerated, Yielded Partial Response in Patients With Advanced Solid Tumors”, Apr. 7, 2013. Therefore, in some embodiments, AZD5363 compositions disclosed herein include 1 mg to 1,000 mg, preferably 100 mg to 750 mg, more preferably, 400 mg to 600 mg twice per day.

2. Molecular Sinks and Peptidomimetics

In some embodiments, the compound that inhibits bioactivity of Akt1, Akt2, or both is a peptide substrate mimic or peptidomimetic that binds to the active site of Akt1 or Akt2 and reduces the bioavailability for its endogenous substrates. Peptide substrate mimic or peptidomimetic can be a fragment of an endogenous substrate of Akt1 or Akt2 that includes the amino acid residue of the endogenous Akt1 or Akt2 substrate that is phosphorylated by Akt1 or Akt2. In some embodiments, the peptide substrate mimic or peptidomimetic can bind to the active site of Akt1 or Akt2 and be phosphorylated by Akt1 or Akt2. In some embodiments, the peptide or peptidomimetic can bind to the active site of Akt1 or Akt2, but cannot be phosphorylated by Akt1 or Akt2. For example, in some embodiments, the peptide or peptidomimetic is fragment of a substrate of Akt1 or Akt2 that includes the residue that is phosphorylated by Akt1 or Akt2, but wherein the residue is mutated to a residue that cannot be phosphorylated. In this way, peptide substrate mimics and peptidomimetics serve as a molecular sink for Akt1 or Akt2 or both and reduce its bioavailability for its endogenous substrates. A broad range of substrates for Akts have been identified, including, but not limited to, transcription factors (e.g. FOXO1), kinases (GSK-3, Raf-1, ASK, Chk1) and other proteins with important signaling roles (e.g. Bad, MDM2).

C. Inhibitory Nucleic Acids for Antagonizing Akt1 and Akt2

Inhibitory nucleic acids can be used to antagonize Akt1, Akt2 or both by inhibiting or down regulating expression of their mRNA. Thus, in some embodiments, the antagonist is an inhibitory nucleic acid that silences gene expression. The nucleic acid sequences for Akt1 and Akt2 are known in the art.

Inhibitory nucleic acid technologies are known in the art and include, but are not limited to, antisense oligonucleotides, catalytic nucleic acids such as ribozymes and deoxyribozymes, aptamers, triplex forming nucleic acids, external guide sequences, and RNA interference molecules (RNAi), particularly small nucleic acid molecules, such as short interfering nucleic acid (siNA), short interfering RNA (siRNA), double-stranded RNA (dsRNA), micro-RNA (mRNA), and short hairpin RNA (shRNA) molecules capable of mediating RNA interference (RNAi).

1. RNA Interference

Gene silencing by RNAi was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

In some embodiments the inhibitory nucleic acid is an siRNA. SiRNA is typically a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. Sequence specific gene or isoform specific silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

Small RNAs include microRNAs (miRNA) and small interfering RNAs (siRNAs). MiRNAs are produced by the cleavage of short stem-loop precursors by Dicer-like enzymes; whereas, siRNAs are produced by the cleavage of long double-stranded RNA molecules. MiRNAs are single-stranded, whereas siRNAs are double-stranded. Therefore, the double-stranded structure may be formed by a single self-complementary RNA strand or two separate complementary RNA strands. RNA duplex formation may be initiated either inside or outside the plant cell.

Suitable inhibitory nucleic acids can contain one or more modified bases, or have a modified backbone to increase stability or for other reasons. For example, the phosphodiester linkages of natural RNA may be modified to include at least one of a nitrogen or sulfur heteroatom. Moreover, nucleic acids comprising unusual bases, such as inosine, or modified bases, such as tritylated bases, to name just two examples, can be used. It will be appreciated that a great variety of modifications have been made to nucleic acids that serve many useful purposes. The term nucleic acids as it is employed herein embraces such chemically, enzymatically or metabolically modified forms of nucleic acids, provided that it is derived from an endogenous template.

The sequence of at least one strand of the RNAi molecule contains a region complementary to at least a part of the target mRNA sufficient for the RNAi molecule to specifically hybridize to the target mRNA. In one embodiment, one strand of the RNAi molecule is substantially identical to at least a portion of the target mRNA.

In one embodiment, the inhibitory nucleic acid has 100% sequence identity with at least a part of the target mRNA. However, inhibitory nucleic acids having 70%, 80% or greater than 90% or 95% sequence identity may be used. Thus sequence variations that might be expected due to genetic mutation, strain polymorphism, or evolutionary divergence can be tolerated.

RNAi molecules includes small RNA molecules which are single stranded or double stranded RNA molecules generally less than 200 nucleotides in length. Such molecules are generally less than 100 nucleotides and usually vary from 10 to 100 nucleotides in length. The duplex region of a double stranded RNA may have a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). While the optimum length of the double stranded RNA may vary according to the target sequence and experimental conditions, the duplex region of the RNA may be at least 19, 20, 21, 22, 23, 25, 50, 100, 200, 300, 400 or more nucleotides long. In a preferred format, small RNA molecules, such as siRNA and shRNA have 11, 12, 13, 14, 15, 16, 17, 18, 19, 20, 21, 22, 23, 24, 25, 26, 27, 28, 29 or 30 nucleotides. Preferably, the nucleotides are contiguous, consecutive nucleotides of complementary to a target mRNA sequence, for example Atk3 mRNA.

In vivo, the RNAi molecule may be synthesized using recombinant techniques well known in the art (see e.g., Sambrook, et al., Molecular Cloning; A Laboratory Manual, Third Edition (2001)). For example, bacterial cells can be transformed with an expression vector which comprises the DNA template from which double stranded RNA is to be derived. Alternatively, the cells in which inhibition of gene or isoform expression is desired may be transformed with an expression vector or by other means. Bidirectional transcription of one or more copies of the template may be by endogenous RNA polymerase of the transformed cell or by a cloned RNA polymerase (e.g., T3, T7, SP6) coded for by the expression vector or a different expression vector. Inhibition of gene or isoform expression may be targeted by specific transcription in an organ, tissue, or cell type; an environmental condition (e.g. temperature, chemical); and/or engineering transcription at a developmental stage or age, especially when the RNAi molecule is synthesized in vivo. RNAi molecules may also be delivered to specific tissues or cell types using known gene delivery systems. The production of siRNA from a vector is commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. are any shRNA designed as described above based on the sequences for the herein disclosed inflammatory mediators.

2. Aptamers

In some embodiments, a compound that reduces the bioavailability of Akt1, Akt2, or both is an aptamer. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules as well as large molecules, such as reverse transcriptase. Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules are known in the art.

3. Ribozymes

In some embodiments, a compound that reduces the bioavailability of Akt1, Akt2, or both is a ribozyme. Ribozymes are nucleic acid molecules that are capable of catalyzing a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Examples of how to make and use ribozymes to catalyze a variety of different reactions are known in the art.

4. Triplex Forming Nucleic Acids

In some embodiments a compound that reduces the bioavailability of Akt1, Akt2, or both are triplex forming nucleic acids. Triplex forming nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Examples of how to make and use triplex forming molecules to bind a variety of different target molecules are known in the art.

5. External Guide Sequences

In some embodiments a compound that reduces the bioavailability of Akt1, Akt2, or both are external guide sequences (EGSs). EGSs are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Examples of how to make and use EGS molecules to facilitate cleavage of a variety of different target molecules are known in the art.

D. Formulations

Formulations of and pharmaceutical compositions including one or more of the disclosed compounds are provided. Dosage ranges for specific small molecules are discussed above based on pre-clinical and clinical trial data. Generally, dosage levels, for the compounds disclosed herein are between about 0.0001 mg/kg of body weight to about 1,000 mg/kg, more preferably of 0.001 to 500 mg/kg, more preferably 0.01 to 50 mg/kg of body weight daily are administered to mammals. In some embodiments, polypeptides or nucleic acids are administered in a dosage of 0.01 to 50 mg/kg of body weight daily, preferably about 0.1 to 20 mg/kg. In some embodiments, nucleic acid dosages can range from about 0.001 mg to about 1,000 mg, more preferable about 0.01 mg to about 100 mg per administration (e.g., daily; or once, twice, or three times weekly, etc.,)

1. Delivery Vehicles

The active agents can be administered and taken up into the cells of a subject with or without the aid of a delivery vehicle. Appropriate delivery vehicles for the disclosed active agents are known in the art and can be selected to suit the particular active agent. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

The active agent(s) can be incorporated into a delivery vehicle prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C. The release point and/or period of release can be varied as discussed above.

2. Pharmaceutical Compositions

Pharmaceutical compositions including the disclosed compounds, with or without a delivery vehicle, are provided. Pharmaceutical compositions can be for administration by parenteral (intramuscular, intraperitoneal, intravenous (IV) or subcutaneous injection), enteral, transmucosal (nasal, vaginal, rectal, or sublingual), or transdermal (either passively or using iontophoresis or electroporation) routes of administration or using bioerodible inserts and can be formulated in dosage forms appropriate for each route of administration.

In certain embodiments, the compositions are administered locally, for example by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the compositions which is greater than that which can be achieved by systemic administration.

a. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tEudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets andcapsules containing tablets, beads, or granulesetc. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray—congealed or congealed and screened and processed.

Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

c. Formulations for Pulmonary and Mucosal Administration

Active agent(s) and compositions thereof can be applied formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

d. Transdermal

Transdermal formulations may also be prepared. These will typically be ointments, lotions, sprays, or patches, all of which can be prepared using standard technology. Transdermal formulations can include penetration enhancers.

### EXAMPLES

### Materials and Methods

Mice and Reagents

In vitro experiments used pMel-1 mice (B6.Cg-Thy1a/Cy Tg(TcraTcrb)8Rest/J) that carry a rearranged T cell receptor transgene (Vβ13) specific for the mouse homologue (pmel-17) of human (gp100) (10). C57BL/6(H-2b) wild-type (WT), Akt 1 knockout (KO), Akt 2 KO and Akt 3 KO mice were also used. Akt 3 KO mice were a generous gift from Dr. Morris Birnbaum (University of Pennsylvania, Pa.) and Dr. Phillip Dennis (NCI, NIH, MD) and were extensively backcrossed onto WT C57BL/6(H-2b) mice. All other mouse strains were purchased from the Jackson Laboratory. The animals were housed under pathogen-free conditions.

MK-2206 was purchased from Selleckchem. It is a highly selective inhibitor of all Akt isoforms with an IC50 of 8 nM for Akt 1, 12 nM for Akt 2 and 65 nM for Akt3. The inhibitor was used in vitro at an optimized concentration of 0.67 μM/ml. AZD5363 was purchased from Selleckchem. It is a highly specific Akt inhibitor and has an IC50 of 3 nM for Akt 1, 8 nM for Akt 2 and 8 nM for Akt 3. The inhibitor was used in vitro at the optimized concentration of 2.4 μM/ml. Akt kinase 1/2 inhibitor (1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate was purchased from Sigma (St. Louis, Mo.) and has an IC50 of 58 nM for Akt 1 and 210 nM for Akt 2 and only inhibits Akt3 at the concentration of 2.12 mM. The inhibitor was used in vitro at the optimal concentration of 2.2 μM/ml, which is 103 fold lower than the IC50 for Akt3 and therefore ensures specificity for Akt 1 and Akt 2. The inhibitors were titrated, and the doses used showed optimal inhibition with minimal effect on viability. Pan Akt inhibitors were used at doses ensuring the inhibition of all three isoforms.

The gp10025-33 9-mer peptide (KVPRNQDWL (SEQ ID NO:1)) was purchased from ANASPEC (Fermont, Calif.) and used for in vitro activation of pMel-1 splenocytes at a 1 μM/ml concentration. Briefly, GP100 is an enzyme involved in pigment synthesis that is expressed by different melanoma cell lines (including B16) and normal melanocytes.

CD8+ enrichment kits were purchased from Miltenyi (Auburn, Calif.), and CD8 T cells were enriched following the manufacturer's instructions.

Fluorochrome labeled antibodies used for flow cytometry were purchased from BD (San Jose, Calif.).

In Vitro Activation of CD8 T Cells

Tumor antigen-specific CD8 T cells Unfractionated splenocytes from pMel-1 mice were homogenized and stimulated in vitro by gp10025-33 peptide at a 1 μM/ml concentration (day 0). Cells were cultured in RPMI 1640 (Lonza, Allendale, N.J.) supplemented with 10% FBS, penicillin (100 U/mL), streptomycin (100 mg/mL), 0.1% β-mercaptoethanol (Life Technologies, Invitrogen, Carlsbad, Calif.) and IL-2 (100 U/ml) (Peprotech, Rocky Hill, N.J.) at 37° C. with 5% CO2. The cells were cultured with or without MK-2206 (0.67, 2 or 6 μM/ml), AZD5363 (0.27, 0.81 or 2.4 μM/ml) or Akt 1/2 inhibitor (2.2, 6.7, or 20.1 μM/ml). The concentration of the inhibitors was maintained throughout the culture by changing the media every 48-72 hours.

On Days 7, 14 and 21, cells were re-stimulated with gp10025-33 peptide at a 1 μM/ml concentration using feeder cells (irradiated WT splenocytes, 4000 Rads) at 1:1 ratio using the same culture conditions.

TCR Stimulation and Co-Stimulation

CD8 T cells from WT, Akt 1, Akt2, and Akt 3 KO mice were enriched using CD8+ enrichment kits (Miltenyi, Auburn, Calif.) according to the manufacturer's instructions (purity was on average 91%) or CD8+ cells were sorted using FACS ARIA II (BD Biosciences, San Jose, Calif.) (purity >99%).

The cells were then stimulated using feeder cells (irradiated WT splenocytes, 4000 Rads) at 1:1 ratio (day 0) using TCR stimulation (anti-CD3 antibody 1 μg/ml, BD Biosciences, San Jose, Calif.) and co-stimulation (anti-CD28 antibody 2.5 μg/ml BD Biosciences, San Jose, Calif.) in the presence of 100 U/ml IL-2 (Peprotech, Rocky Hill, N.J.). The cells were cultured with or without the optimized doses of the inhibitors MK-2206 at a 0.67 μM/ml concentration or Akt 1/2 inhibitor at a concentration of 2.2 μM/ml, and the concentration was maintained throughout the culture by changing the media every 48-72 hours. Cells were cultured in the same conditions described above.

Proliferation Assay and Phenotyping of CD8 T Cells

Prior to their stimulation (day 0), cells were labeled with 5 μM Violet Cell Trace (VCT) proliferation dye (Life Technologies, Invitrogen, Carlsbad, Calif.) following the manufacturers' instructions. Samples were then evaluated for CD8 T cell expansion via VCT dye dilution (day 3) using an LSRII SORP with HTS Flow Cytometer (BD Biosciences, San Jose, Calif.). The data were analyzed using FlowJo 9 or 10 (Tree Star).

To assess the phenotype of the T cells, the cultured cells were harvested and analyzed on days 3, 7, 14 and 21. The cells were stained with APC-Cy7 labeled anti-CD8, FITC labeled anti-Vβ13, PE labeled anti-CD62L, APC labeled anti-CD44, PE-CF594 labeled anti-CD127, APC labeled anti-KLRG-1 in addition to the viability stain 7AAD (BD Biosciences, San Jose, Calif.). The same APC-Cy7 labeled anti-CD8 was used for sorting the cells using the FACS ARIA II (BD Biosciences, San Jose, Calif.).

For proliferation and phenotyping, the analyses were performed on CD8 T cells specific for the gp100 antigen and were gated on viable (7AAD−), Vβ13+CD8+ T cells. For WT and KO cells, the cells were gated on viable (7AAD−), CD8+ T cells.

Cytometric Bead Array

Following the same activation protocol mentioned above, the pMel-1 gp100-specific CD8 T cells were harvested on day 7 after the first and second stimulation. The viable cells (trypan blue negative) were then counted and co-incubated (at 1:1 ratio) with 1 μM/ml gp10025-33 pulsated irradiated splenocytes (4000 Rads) for 24 hours using the same culture conditions. The supernatant was collected and the level of IL-2, TNF and IFN-γ was assessed using the mouse Th1/Th2/Th17 Cytokine Kit BD™ Cytometric Bead Array (CBA) kit. The data was collected using an LSRII SORP with HTS flow cytometer (BD Biosciences, San Jose, Calif.), and analyzed using the FCAP Array Software v3.0 (BD Biosciences, San Jose, Calif.).

Statistics

All statistical parameters (average values, SD, significant differences between groups) were calculated using GraphPad Prism Software. Statistical significance between groups was determined by paired t test or one-way ANOVA with post hoc Tukey's multiple comparison test (p<0.05 was considered statistically significant).

### Example 1: Akt Inhibition Enhances the Central Memory Phenotype of CD8 T Cells by Diminishing their Terminal Differentiation and Increasing their Proliferative Ability and Survival

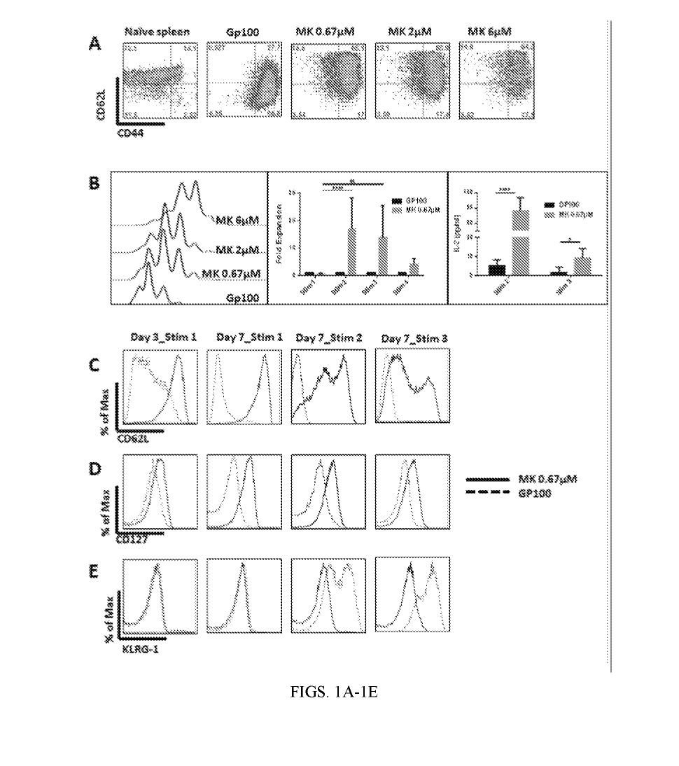

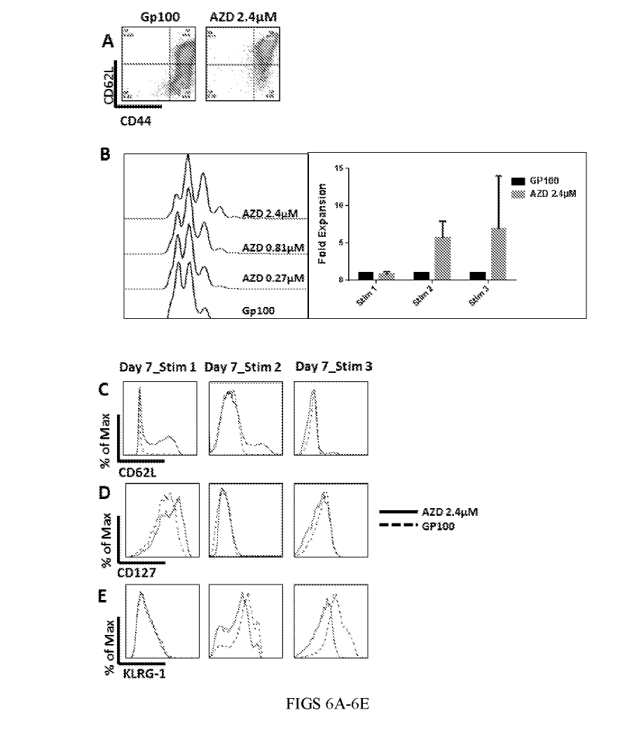

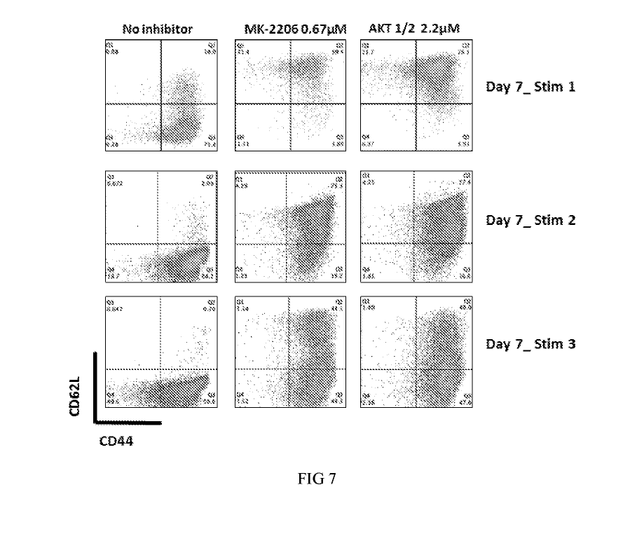

T_{CM} CD8 T cells are superior mediators of therapeutic antitumor immunity due to their greater proliferative capacity upon antigen re-encounter (Klebanoff, et al., Proc Natl Acad Sci USA, 102:9571-9576 (2005); Wherry, et al., Nat Immunol, 4:225-234 (2003); Roberts, et al., J Exp Med, 202:123-133 (2005); Wu, et al., Cancer Lett (2013)). Many T cell functions are governed by PI3K/Akt signaling, including proliferation, survival, migration, and metabolism (Finlay, et al., Ann NY Acad Sci, 1183:149-157 (2010); Kane, et al., Immunol Rev, 192:7-20 (2003)). To test the role of Akt in the differentiation and proliferation of CD8 T cells, the effect of the pan Akt inhibitors MK-2206 and AZD5363 on stimulated CD8 T cells was investigated. This was done using unfractionated splenocytes from pMel-1 mice activated with 1 μM/ml gp10025-33. The phenotype of CD8 T cells was assessed after 3 days of stimulation. It was found that MK-2206-treated cells consisted mainly of T_{CM} cells (CD62LhiCD44hi) and displayed a higher percentage of naïve cells (CD62LhiCD44lo) (FIG. 1A). On the other hand, the majority of non-MK-2206-treated cells were TEM cells (CD62LloCD44hi). This was observed at all the concentrations used (FIG. 1A). The same pattern was also detected in AZD5363-treated cells (FIG. 6A). This shows that Akt inhibition retards the terminal differentiation of CD8 T cells and holds them in the central memory and earlier differentiation stages. The same effect was seen after the second and third stimulation with gp10025-33 on days 7, 14 and 21 in the presence of the Akt inhibitors (FIG. 7 and data not shown). This shows that Akt inhibition preserves a healthy reservoir of T_{CM} cells even after several encounters with the antigen.

Because T_{CM} CD8 T cells are known to possess a greater proliferative ability than TEM cells upon antigen re-encounter (Klebanoff, et al., Proc Natl Acad Sci USA, 102:9571-9576 (2005); Wherry, et al., Nat Immunol, 4:225-234 (2003); Roberts, et al., J Exp Med, 202:123-133 (2005); Wu, et al., Cancer Lett (2013)), the proliferation of CD8 T cells was then assessed. Following the first three days of stimulation (to differentiate antigen-specific CD8 T cells) it was found that the proliferation of CD8 T cells treated with MK-2206 or AZD5363 was inhibited in a dose-dependent manner as measured by VCT dilution, as expected (FIG. 1B and FIG. 6B). However, surprisingly, with further stimulation (on days 7, 14 and 21), the MK-2206-treated CD8 T cells were found to expand at a significantly higher rate than the non-treated cells (FIG. 1B). On the other hand, the non-treated cells lost the ability to expand following the third stimulation. This clearly shows that Akt inhibition leads to enhanced longevity and prolonged cell survival of the CD8 T cells. The same effect was observed with AZD5363 (FIG. 6B).

Interestingly, it was found that CD8 T cells treated with MK-2206 maintained high expression levels of CD62L and CD127 (markers associated with high proliferative potential). This correlates with the enhanced proliferation ability of the T_{CM} cells treated with the inhibitor. These high levels were observed on days 3, 7, 14 and 21 (FIGS. 1C and D).

The ability of CD8 T cells to proliferate was further assessed by measuring the level of IL-2 secretion, which is diminished in terminally differentiated CD8 T cells. It was found that CD8 T cells treated with MK-2206 maintained a significantly high level of IL-2 secretion when re-stimulated on days 7 and 14 (FIG. 1B).

The increase in longevity and survival observed in the MK-2206-treated CD8 T cells prompted us to assess the expression level of the exhaustion marker KLRG-1, which is up-regulated in terminally differentiated cells. It was found that Akt inhibition maintained a low level of KLRG-1 after the second and third stimulations, whereas the non-treated CD8 T cells expressed a significantly higher level of this exhaustion marker (FIG. 1E). The ability of MK-2206 to delay the exhaustion of CD8 T cells corresponds to the cells' ability to survive and expand in response to more stimulations than the non-treated cells (FIG. 1B). The same effect on activation and exhaustion markers was observed when cells were treated with the Akt inhibitor AZD5363 (FIGS. 6C-6E).

Taken together, these data show that Akt inhibition preserves T_{CM} cells, hence enhancing their proliferative potential and survival while delaying the terminal differentiation and exhaustion of CD8 T cells.

### Example 2: Akt Inhibition Rescues the Ability of CD8 T Cells to Produce Cytotoxic Cytokines after Multiple Stimulations

Targeting Akt using pan Akt inhibitors enhances proliferation, preserves the T_{CM} phenotype, delays exhaustion and conserves a larger pool of naïve cells. To assess the function of T_{CM} cells, the secretion levels of IFN-γ and TNF were tested.

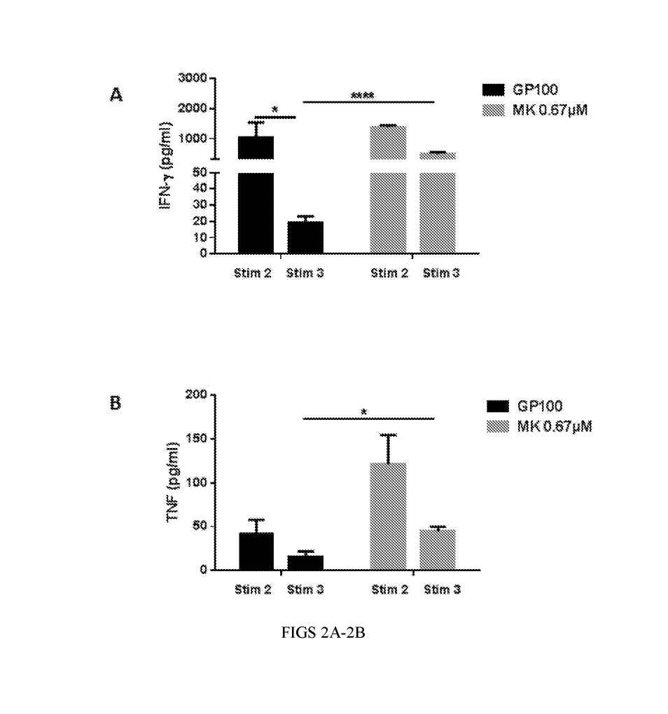

CD8 T cells were re-stimulated on days 7 and 14 with gp100₂₅₋₃₃ and the level of IFN-γ and TNF production after 24 hours was assessed. After the second stimulation, MK-2206-treated and non-treated cells produced high and comparable levels of IFN-γ and TNF in response to antigen reencounter (FIG. 2A). After the third stimulation, the secretion of IFN-γ and TNF dropped significantly; however, Akt inhibition rescued the ability of CD8 T cells to produce these cytokines, as their ability to secrete IFN-γ and TNF was maintained at a significantly higher level (FIG. 2A). This suggests that CD8 T cells undergo terminal differentiation and reach exhaustion, thus losing their ability to secrete IFN-γ and TNF upon several encounters with the antigen. Akt inhibition can clearly rescue the ability of CD8 T cells to produce cytotoxic cytokines even with further stimulation.

The maintained levels of IFN-γ and TNF secretion suggest that CD8 T cells do not lose their cytotoxic functionality as a result of Akt inhibition. In fact, Akt inhibition enhances their proliferative ability and survival by delaying their terminal differentiation and prolongs their ability to produce cytotoxic cytokines.

### Example 3: Akt 1 and Akt 2 are the Two Isoforms Responsible for Terminal Differentiation of CD8 T Cells

Akt inhibition in CD8 T cells delays their terminal differentiation, preserves T_{CM} cells, enhances their proliferative ability and cytokine secretion and prolongs their survival. The role of specific Akt isoforms (Akt1, Akt2 and Akt3) in the development, proliferation and function of CD8 T cells is only known during thymic development, where Akt1 and Akt2 are the main isoforms contributing to the transition towards the double positive (CD4+CD8+) stage and are involved in the differentiation of single positive T cells (Mao, et al., J Immunol, 178:5443-5453 (2007); Juntilla, et al., Proc Natl Acad Sci USA, 104:12105-12110 (2007)). To further dissect the role of specific Akt isoforms in the differentiation and proliferation of CD8 T cells, we tested the effect of Akt 1 and Akt 2 inhibition using an Akt 1/2 inhibitor on stimulated CD8 T cells. This was done using unfractionated splenocytes from pMel-1 mice activated with 1 μM/ml gp100₂₅₋₃₃.

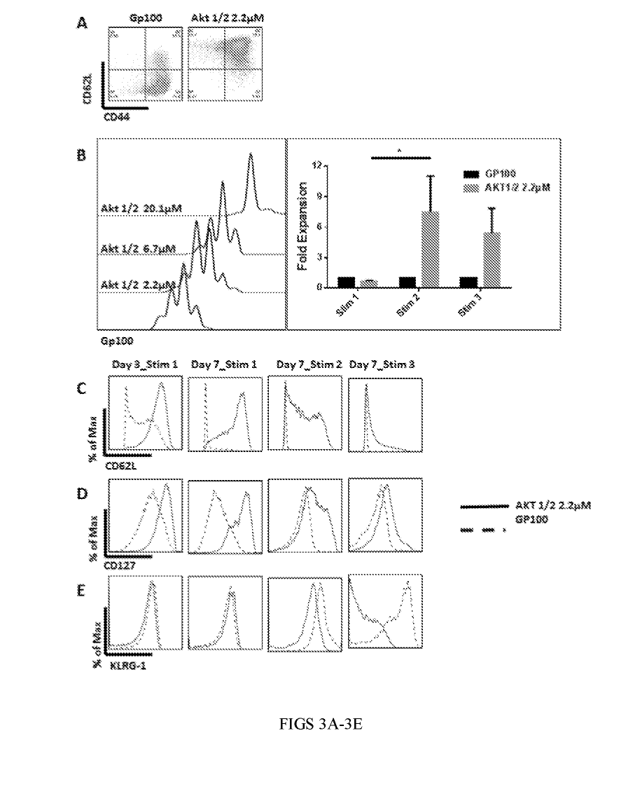

The phenotype of the cells was assessed after 3 days of stimulation. Similar to what we observed with the pan Akt inhibitors, CD8 T cells treated with Akt1/2 inhibitor consisted mainly of T_{CM} cells and displayed a higher percentage of naïve cells (FIG. 3A). On the other hand, the majority of the non-treated CD8 T cells were TEM cells. This effect was persistent even after the second and third stimulations (FIG. 7). These findings suggest that Akt1 and Akt2 are the isoforms responsible for terminal differentiation of CD8 T cells and that their inhibition maintains CD8 T cells in earlier stages of differentiation (naïve and T_{CM}).

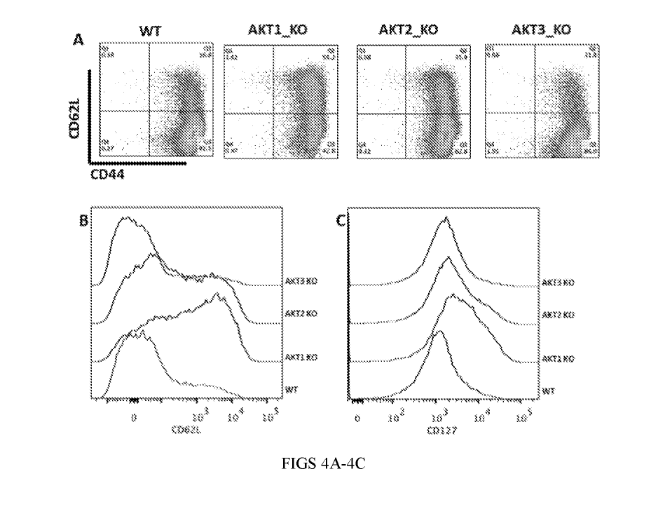

To test if the TCM phenotype generated by the inhibition of Akt1 and Akt2 possesses an enhanced proliferative ability, the proliferation of CD8 T cells was assessed after 3 days of stimulation. It was found that the proliferation of CD8 T cells treated with Akt 1/2 inhibitor was inhibited in a dose-dependent manner (FIG. 3B). Additionally, the inhibition of Akt1 and Akt2 significantly enhanced the proliferative ability of CD8 T cells with further stimulations (days 7 and 14) (FIG. 4A). Treatment of CD8 T cells with Akt 1/2 inhibitor also maintained high expression levels of CD62L and CD127 (FIGS. 3C and D) and high secretion levels of IL-2 (data not shown), consistent with the enhanced proliferative ability of TCM cells.

Similar to what we observed with pan Akt inhibitors, treatment of CD8 T cells with Akt 1/2 inhibitor prolonged their survival and delayed their exhaustion as evidenced by the maintained low levels of KLRG-1 following multiple stimulations (FIG. 3E). Treating the cells with Akt 1/2 inhibitor also preserved high levels of TNF and IFNγ secretion (data not shown), suggesting an enhanced cytotoxic functionality of CD8 T cells.

To rule out any influence of non-specific inhibition of different isoforms and kinases using Akt inhibitors, the phenotype of stimulated CD8 T cells from Akt 1, 2 and 3 KO and WT mice was assessed. After 7 days of stimulation, the highest percentage of TCM cells was observed from Akt1 KO mice, followed by Akt2 KO CD8 T cells. CD8 T cells from both WT and Akt3 KO mice had comparable levels of TCM cells, which were significantly lower than Akt1 and 2 KO CD8 T cells (FIG. 4A). Additionally, CD8 T cells from Akt1 and Akt2 KO mice displayed a higher percentage of naïve cells in comparison to cells from WT and Akt3 KO mice (FIG. 4A). Akt1 KO cells expressed a higher level of CD62L and CD127 than Akt2 KO, which in turn expressed higher levels of these markers than WT and Akt3 KO cells (FIG. 4B, C). However, CD8 T cells from WT and from different Akt KO mice did not show any differences in proliferation after several stimulations (data not shown). This could be explained by compensation from the different isoforms once the CD8 T cells are stimulated in vitro.

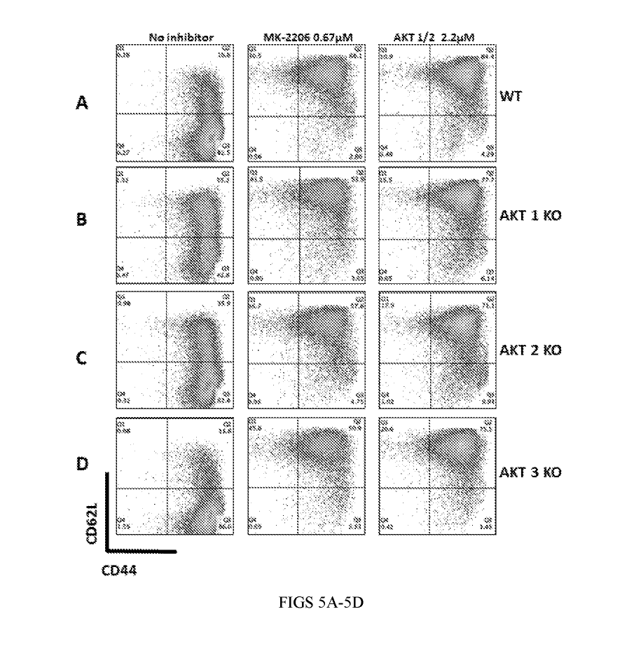

To assess the contribution of each Akt isoform to the differentiation of TCM cells, stimulated CD8 T cells from WT and Akt KO mice were treated with the pan Akt and Akt1/2 inhibitors. Interestingly, cells from all the Akt KO mice and from WT mice behaved similarly, where the inhibition of Akt resulted in the enhancement of T_{CM} phenotype (FIG. 5). In Akt1 and Akt2 KO cells, the inhibitors target the existing isoform, thus enhancing the effect of the absent isoform. Therefore, TCM cells from Akt1 and Akt2 KO mice are maintained at comparable levels to WT treated with the same inhibitors. On the other hand, the absence of Akt3 in the KO mice did not affect the differentiation of CD8 T cells, and the inhibition of the existing isoforms (Akt1 and Akt2) resulted in a similar outcome to Akt inhibition in WT mice. This finding confirms that Akt3 does not play any significant role in the development of TCM cells.

Taken together, our data demonstrates that the inhibition of Akt1 and Akt2, but not Akt 3, mitigates the terminal differentiation of CD8 T cells and preserves T_{CM} cells, thus enhancing their proliferative potential, longevity, and survival and rescues their ability to produce cytokines.

During the T cell response, CD8 T cells comprise effector and memory T cells (Kim, et al., Front Immunol, 4:20 (2013); Klebanoff, et al., Immunol Rev, 211:214-224 (2006)). CD8 memory T cells can be classified into T_{CM} and T_{EM} cells (Klebanoff, et al., Immunol Rev, 211:214-224 (2006); Sallusto, et al., Nature, 401:708-712 (1999)). TCM cells are superior in their ability to protect against viral and bacterial challenges (Klebanoff, et al., Proc Natl Acad Sci USA, 102:9571-9576 (2005); Wherry, et al., Nat Immunol, 4:225-234 (2003)) and mediation of therapeutic antitumor immunity when compared to TEM cells, due to their greater proliferative capacity upon antigen re-encounter (Klebanoff, et al., Proc Natl Acad Sci USA, 102:9571-9576 (2005); Wherry, et al., Nat Immunol, 4:225-234 (2003); Roberts, et al., J Exp Med, 202:123-133 (2005); Wu, et al., Cancer Lett (2013)).

Multiple T cell functions are governed by PI3K/Akt signaling, including proliferation, survival, migration, and metabolism (Finlay, et al., Ann NY Acad Sci, 1183:149-157 (2010); Kane, et al., Immunol Rev, 192:7-20 (2003)). In fact, the differentiation of CD8 cells into memory T cells is thought to be coordinated, at least in part, by the PI3K/Akt pathway (Kim, et al., Front Immunol, 4:20 (2013); Li, et al., J Immunol, 188:3080-3087 (2012); Araki, et al., Nature, 460:108-112 (2009)). Inhibition of Akt in vivo increases the number of memory CD8 T cells (Kim, et al., J Immunol, 188:4305-4314 (2012)), and the downstream inhibition of mTOR augments the functional quality of cytotoxic CD8 T cell responses by prompting a CD8 memory phenotype (Li, et al., J Immunol, 188:3080-3087 (2012); Araki, et al., Nature, 460:108-112 (2009); Mineharu, et al., Mol Cancer Ther (2014)). However, mechanisms that enhance the TCM phenotype and delay the terminal differentiation of CD8 T cells without significantly impacting their proliferation and function are still needed.

The data provided herein show for the first time, that the Akt1 and Akt2 isoforms drive the terminal differentiation of antigen specific CD8 T cells. We further report that the inhibition of Akt1 and Akt2 delays the exhaustion of CD8 T cells, prolongs their survival, preserves a remarkably high percentage of TCM cells, and significantly increases their proliferative potential upon reencountering antigen.

The data show that Akt1 and Akt2 inhibition enhances the proliferative potential of CD8 cells and maintains a high expression of CD62L and CD127, while mitigating their exhaustion as evidenced by the low expression level of KLRG-1 and reduction in the expression of PD-1 (data not shown).

Interestingly, inhibition of mTOR by rapamycin has been shown to enhance the memory phenotype of CD8 T cells as a result of modulating the functional quality of CD8 T cells rather than their proliferation (Li, et al., J Immunol, 188:3080-3087 (2012); Araki, et al., Nature, 460:108-112 (2009); Mineharu, et al., Mol Cancer Ther (2014)). In fact, in the tumor micro-environment, mTOR inhibition leads to a decrease in the proliferation of CD8 T cells by direct inhibition coupled with a significant increase in the suppressive regulatory T cell (Treg) population (Kim, et al., Oncoimmunology, 3:e29081 (2014)). Rapamycin has been shown to support the proliferation and survival of Treg cells (Long, et al., J Autoimmun, 30:293-302 (2008); Strauss, et al., J Immunol, 178: 320-329 (2007); Battaglia, et al., Blood, 105:4743-4748 (2005); Basu, et al., J Immunol, 180:5794-5798 (2008)) due to a feedback loop where mTOR inhibition results in PI3K-dependent Akt activation, which sustains signaling through mTOR (Sun, et al., Cancer Res, 65:7052-7058 (2005)). Accordingly, antibody-based depletion of Treg cells was proposed recently as a strategy to counteract this inhibition of CD8 T cells when rapamycin is used to augment memory T cells (Kim, et al., Oncoimmunology, 3:e29081 (2014)). While this suggested strategy can minimize the suppression of CD8 T cells by Tregs, it does not overcome the direct proliferation inhibition exerted on CD8 cells by rapamycin. Here, we demonstrate that inhibition of the PI3K/Akt pathway at the level of Akt significantly enhances the proliferation of memory CD8 T cells. Furthermore, it was recently shown that PI3K/Akt pathway inhibitors selectively target Tregs and result in a significant enhancement of anti-tumor immune response, including a significant decrease in Treg cells and increase in CD8 T cells within the tumor micro-environment (Abu-Eid, et al., Cancer Immunol Res (2014)). Therefore, using Akt inhibitors can simultaneously enhance the effector arm by augmenting the memory CD8 T cells and diminish the suppressive arm by selectively inhibiting Treg cells.

CD8 T cells progressively lose IL-2 production as a function of differentiation from naïve to effector cells (Sallusto, et al., Nature, 401:708-712 (1999)). Akt inhibition in CD8 T cells maintains a significantly higher level of IL-2 secretion. This is consistent with their maintained ability to proliferate and their phenotype as TCM cells. Remarkably, the enhanced proliferative ability of CD8 T cells exerted by Akt inhibition is isoform-specific, thus suggesting the possibility of precise targeting of Akt1 and Akt2 to modulate the CD8 T cell response with minimal effects on other cellular functions.

Additionally, Akt1 and Akt 2 inhibition can rescue the ability of CD8 T cells to secrete high levels of TNF and IFNγ secretion, even following multiple stimulations, thus suggesting a prolonged and potent anti-tumor cytotoxic ability. This suggests the use of Akt isoform-specific inhibitors to produce a sustained and powerful anti-tumor T cell response when combined with different cancer immune therapies.

The data shows that Akt1 and Akt2 inhibition leads to the preservation of a percentage of naïve CD8 T cells. It has been shown that naïve CD8 T cells have greater proliferative potential (Wen, et al., Chin Med J (Engl), 127:1328-1333 (2014); Hinrichs, et al., Blood, 117:808-814 (2011)) and display elevated levels of IL-2 and IFNγ secretion following secondary stimulations (Wen, et al., Chin Med J (Engl), 127:1328-1333 (2014)). Additionally, effector T cells derived from naïve T cells were found to promote more potent in vivo anti-tumor activity (Wen, et al., Chin Med J (Engl), 127:1328-1333 (2014); Hinrichs, et al., Blood, 117:808-814 (2011)). Hence, our data show that Akt1 and Akt2 inhibition maintains a higher percentage of both naïve and TCM cells, which are both superior mediators of anti-tumor activity in comparison to effector and TEM CD8 T cells (Klebanoff, et al., Proc Natl Acad Sci USA, 102:9571-9576 (2005); Wherry, et al., Nat Immunol, 4:225-234 (2003); Roberts, et al., J Exp Med, 202:123-133 (2005); Wu, et al., Cancer Lett (2013); Wen, et al., Chin Med J (Engl), 127:1328-1333 (2014); Hinrichs, et al., Blood, 117:808-814 (2011)).

To rule out any influence of undesired inhibition of different isoforms and different kinases using Akt inhibitors, the phenotype of CD8 T cells from different Akt isoform KO mice was assessed. The data confirm that the complete absence of Akt 1, and to a lesser extent Akt 2, preserves a larger reservoir of T_{CM} cells when compared to WT CD8 T cells. Furthermore, the absence of Akt 3 has no effect on the percentage of TCM cells and is comparable to cells from WT mice.

This suggests that in CD8 T cells, Akt 1 and Akt 2 push towards the more powerful and acute T_{EM} response. While this is the desired situation in cases of acute infections, a continuous exposure to antigen in cases of chronic infections and tumors leads to depletion of the T_{EM} cells and the small reservoir of T_{CM} CD8 T cells. The inhibition of Akt1 and Akt2 isoforms seems to reverse this effect and favors a more sustainable response without affecting the number or function of available CD8 T cells.

Akt1 and Akt2, but not Akt3, drive the terminal differentiation of CD8 T cells and that their inhibition enhances the T_{CM} phenotype, improves CD8 T cell survival, prolongs their cytokine production ability and enhances their proliferative potential.

To date, mechanisms that slow down terminal differentiation of CD8 T cells without substantially impacting proliferation after TCR stimulation are still lacking. A mechanism in which proliferative potential, function and survival are enhanced by maintaining a reservoir of T_{CM} and naïve cells using only Akt1 and Akt2 inhibition is provided. These findings strongly suggest the utility of using Akt isoform inhibitors to modulate the immune response as part of cancer immune therapy.

We claim:

1. A method for maintaining a reservoir of central T memory cells in a subject comprising:
administering to the subject an effective amount of one or more inhibitors of Akt1 and Akt2 to enhance the proliferative potential, function, and survival of CD8 T cells.

2. (canceled)

3. A method for reducing T cell exhaustion in a subject comprising administering and effective amount of one or more inhibitors of Akt1 and Akt2 to enhance the central memory phenotype of CD8 T cells by diminishing their terminal differentiation and increasing their proliferative ability and survival.

4. A method for treating cancer or a tumor in a subject comprising administering to the subject in need thereof an effective amount of one or more inhibitors of Akt1 and Akt2 to enhance the central memory phenotype of CD8 T cells by diminishing their terminal differentiation and increasing their proliferative ability and survival.

5. (canceled)

6. A method for maintaining secretion of TNF and IFNγ by CD8 T cells in a subject comprising administering to the subject an effective amount of one or more inhibitors of Akt1 and Akt2 to maintain secretion of high levels of TNF and IFNγ by CD8 T cells following multiple stimulations.

7.-8.

9. A method for maintaining expression of CD62L and CD127 on CD8 cells of a subject comprising administering to the subject an effective amount of one or more inhibitors of Akt1 and Akt2 to maintain a high expression of CD62L and CD 127 on CD8 T cells in the subject.

10. The method of claim 1, wherein the one or more inhibitors are selected from the group consisting or MK-2206, AZD5363, (1,3-Dihy dro-1-(1-((4-(6-pheny 1-1H-imidazo[4,5-g] quinoxalin-7-yl)phenyl)methyl)-4-piperidinyl)-2H-benzimidazol-2-one trifluoroacetate salt hydrate or combinations thereof.

11. The method of claim 1, wherein the inhibitors is selected from the group consisting of an antibody, antisense oligonucleotide, siRNA, microRNA, aptamer, and external guide sequence.

12. The method of claim 1, wherein the subject is also administered a second therapeutic agent.

13. The method of claim 12, wherein the second therapeutic agent is selected from the group consisting of T cells genetically engineered to produce chimeric antigen receptors, immunostimulatory agents, chemotherapeutics, stem cells, and antibodies.

14. The method of claim 1, wherein the subject is human.

15. A pharmaceutical composition comprising an effective amount of an inhibitor of Akt1 and an effective amount of Akt2 to enhance the proliferative potential, function, and survival of CD8 T cells when administered to a subject.
